(19) United States
(12) Patent Application Publication
(10) Pub. No.: US 2026/0263525 A1
(43) Pub. Date: Sep. 10, 2026

(54) PROTOCOL FOR GENERATING NEURAL PROGENITOR CELLS AND IMPLEMENTATIONS THEREOF

(71) Applicant: Eyestem Research Private Limited, Karnataka, Bengaluru (IN)

(72) Inventors: Rajarshi PAL, Karnataka, Bengaluru (IN); Rashmi Theresa PRABHU, Lonavla, Maharashtra (IN); Swapna NANDAKUMAR, Karnataka, Bengaluru (IN); Swathi NARASIMHAIAH, Karnataka, Bengaluru (IN); Harshini SURENDRAN, Tamilnadu Coimbatore (IN); Indrani DATTA, Karnataka, Bengaluru (IN); Kallolika MONDAL, Kalyani Nadia West Bengal (IN)

(21) Appl. No.: 19/108,615

(22) PCT Filed: Sep. 5, 2023
(86) PCT No.: PCT/IN2023/050835

§ 371 Date: Mar. 4, 2025

(30) Foreign Application Priority Data
Sep. 5, 2022 (IN) 202241050601
Sep. 30, 2022 (IN) 202241056369

(51) Int. Cl. A61K 35/30; A61K 9/00; A61P 25/16; A61P 25/28; C12N 5/00; C12N 5/0793

(52) U.S. Cl. CPC A61K 35/30; A61K 9/0043; A61P 25/16; A61P 25/28; C12N 5/0068; C12N 5/0619; C12N 2501/15; C12N 2501/415; C12N 2506/45; C12N 2509/00; C12N 2513/00; C12N 2533/32; C12N 2533/52; C12N 2533/54; C12N 2533/90; C12N 2539/00

(57) ABSTRACT

A cell culture protocol for obtaining neural progenitor cells from induced pluripotent stem cells is described. Also described are a pharmaceutical composition and a medicament containing the neural progenitor stem cells for use in the treatment of neuro degenerative disorders such as Parkinson's disease as described.

### FIELD OF INVENTION

The present disclosure relates to the field of cell culture techniques and cell differentiation protocols in general and cell culture protocol for producing neural progenitor cells from induced pluripotent stem cells in particular.

### BACKGROUND OF INVENTION

Neurological disorders are diseases of the central and peripheral nervous system, affecting millions of people worldwide. Neurodegenerative disorders occur when nerve cells lose function over time and ultimately die. Alzheimer disease and, Parkinson's disease, are two of the most common neurological disorders, whose prevalence and incidence rise dramatically with age. The incidence of these neurological disorders is expected to continually increase, with the increasing life expectancy in many countries around the world.

Parkinson disease (PD) is a progressive neurodegenerative disorder. Resting tremor, bradykinesia, rigidity, and postural instability are the major motor manifestations of PD. Accumulation of α-synuclein-positive protein aggregates in surviving neurons, termed Lewy bodies, and loss of nigrostriatal dopaminergic neurons are the main pathological hallmarks (Antony P M, Diederich N J, Krüger R, Balling R. The hallmarks of Parkinson's disease. FEBS J. 2013 December; 280 (23): 5981-93. doi: 10.1111/febs.12335. Epub 2013 Jun. 10). Motor symptoms in PD are treated using L-DOPA, dopamine agonists, and monoamine oxidase and catechol-O-methyltransferase inhibitors. These treatment strategies are intended to supplement the depleting dopamine levels. However, these interventional strategies for PD mainly focus on symptomatic relief and offer little toward prevention of disease progression.

Since dopaminergic cell loss is the main hallmark of PD, cell replacement strategies have been in consideration as potential therapy (Armstrong, M. J.; Okun, M. S. Diagnosis and Treatment of Parkinson Disease: A Review. JAMA 2020, 323 (6), 548-560). Early studies on transplantation strategies for PD utilized fetal midbrain tissues that demonstrated modest recovery in younger patients but no recovery in older patients. However, transplantation of fetal tissues is accompanied by ethical challenges, and high variability in tissue specimens that significantly affects clinical outcomes. Stem cell therapy, as an alternative to fetal-derived tissues, is the current mainstay for all cell replacement strategies across the whole spectrum of degenerative diseases. Given that the goal of cell therapy is replacement of dopaminergic neurons, several studies have outlined strategies for the derivation of dopaminergic neurons.

U.S. Pat. No. 11,236,302B2 describes a method for producing dopaminergic cells from human pluripotent stem cells and further, describes the use of the dopaminergic cells for treating neurodegenerative diseases including Parkinson's disease.

Despite the availability of several methods, generating pure populations of dopaminergic neurons in sufficient numbers continues to remain a challenge. As in vitro derivation strategies result in a mixed pool of cells, neural progenitor cells (NPCs) can be considered a promising alternative to terminally differentiated dopaminergic neurons.

With increasing cases of neurodegenerative disorders, there is an immediate need to explore efficient cell culture protocols to generate NPCs and thereby assess their therapeutic potential in treating such neurodegenerative disorders.

### SUMMARY OF THE INVENTION

In an aspect of the present disclosure, there is provided a cell culture protocol for obtaining NPCs (neural progenitor cells) from iPSCs (induced pluripotent stem cells) comprising the steps of: (a) generating embryoid bodies from a confluent culture of iPSCs, wherein the embryoid bodies are in non-adherent suspension culture, (b) plating the embryoid bodies on a culture dish coated with a suitable extracellular matrix in neural induction media (NIM) to obtain neuroectoderm lineage, wherein the NIM comprises at least one WNT pathway inhibitor and at least two SMAD pathway inhibitors, (c) culturing the neuroectoderm lineage for 6-8 days in neural proliferation media (NPM) for primary rosette formation, wherein the NPM does not comprise any inhibitor, (d) selecting and plating the primary rosettes as suspension culture on ultra-low attachment culture dish, (e) culturing the primary rosettes of step (d) in suspension culture for 4-5 days to facilitate primary neurosphere formation, (f) plating the primary neurospheres of step (e) on a culture dish coated with suitable extracellular matrix to obtain adherent primary neurospheres, (g) culturing the adherent primary neurospheres in the NPM for 5-6 days for secondary rosette formation, (h) selecting and plating the secondary rosettes as suspension culture on ultra-low attachment dish, (i) culturing the secondary rosettes of step (h) in suspension culture for 4-5 days to facilitate secondary neurosphere formation, (j) plating the secondary neurospheres on a culture dish coated with suitable extracellular matrix to obtain adherent secondary neurospheres, (k) culturing the adherent secondary neurospheres in the NPM for 5-7 days for tertiary rosette formation, and (l) enzymatically dissociating and plating the tertiary rosette to obtain the NPCs.

In another aspect of the present disclosure, there is provided a pharmaceutical composition comprising the neural progenitor cells obtained from a method as described herein and pharmaceutically acceptable excipients.

In another aspect of the present disclosure, there is provided a method of treating a neurodegenerative disorder in a subject comprising administering the nasal formulation as described herein to the subject.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1A-1D illustrates the phase contrast images displaying temporal changes in cellular morphology from undifferentiated iPSCs to neural progenitor cells, in accordance with an embodiment of the present disclosure.

FIG. 2A-2F depicts the immunocytochemistry images of neural progenitor cells with different cell markers such as A) SOX1, B) SOX2, C) PAX6, D) TUJ1, E) NESTIN and OCT4, F) SSEA1 and Ki67 and, in accordance with an embodiment of the present disclosure.

FIG. 3A-3C depicts the quantitative analysis of cell markers signifying the identity and purity of neural progenitor cells. 3A-3C depicts the RT-PCR results of gene expression profile of different cell markers of neural progenitor cells on differentiation, in accordance with an embodiment of the present disclosure.

FIG. 4A-4C depicts the quantitative analysis of cell markers signifying the identity and purity of neural progenitor cells. 4A-4C depicts the flow cytometry results of protein expression profile of different cell biomarkers of neural progenitor cells on differentiation, in accordance with an embodiment of the present disclosure.

FIG. 5A-5B depicts the differential gene expression analysis results of NPCs. FIG. 5A is an interactive heat map plot for the top 20 upregulated and down regulated genes for Control Vs NPC-B3 cells. Gradient scale is based on expression.

FIG. 5B depicts the volcano plot of the differential gene expression analysis for Control Vs NPC-B3 cells. The plot displays the log 2 differential gene expression analysis where the green points indicate significantly upregulated genes, and the red points indicate significantly down regulated genes, in accordance with an embodiment of the present disclosure.

FIG. 6A-6B depicts the mRNA sequencing analysis results of NPCs. FIG. 6A depicts the top enriched biological processes for upregulated genes based on p-value in Control Vs NPC-B3 cells. FIG. 6B depicts the splice junction distribution of the samples in the novel, known and partly novel regions in Control Vs NPC-B3 cells, in accordance with an embodiment of the present disclosure.

FIG. 7 illustrates the experimental plan for assessing the potential of NPCs in rescuing a Parkinson's disease induced in vivo model, in accordance with an embodiment of the present disclosure.

FIG. 8A-8B depicts the biodistribution of NPCs in different regions of the brain in Control vs. Parkinson's disease induced in vivo model for 1 hour, 3 hour and 24 hour post transplantation of NPCs, in accordance with an embodiment of the present disclosure.

FIG. 9A-9B depicts the biodistribution of NPCs in different regions of the brain in Parkinson's disease induced in vivo model for 1 hour, 3 hour, 24 hour, 72 hour and 10 days post transplantation of NPCs, in accordance with an embodiment of the present disclosure.

FIG. 10A-10B depicts the biodistribution of NPCs in other blood filtering organs in Parkinson's disease induced in vivo model for 1 hour, 3 hour, and 24 hour, post transplantation of NPCs, in accordance with an embodiment of the present disclosure.

FIG. 11A-11B depicts the biodistribution of NPCs in other blood filtering organs in Parkinson's disease induced in vivo model for 1 hour, 3 hour, 24 hour, 72 hour and 10 days post transplantation of NPCs, in accordance with an embodiment of the present disclosure.

FIG. 12A-12B illustrates the behavioral changes observed in Parkinson's disease induced in vivo model vs. control post transplantation of NPCs, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

Neural progenitor cells (NPCs) are the progenitor cells of the central nervous system (CNS) that give rise to many, if not all, of the glial and neuronal cell types that populate the CNS. For the purpose of the present disclosure the abbreviations “NPCs” and “NPs” are used interchangeably and used to denote neural progenitor cells.

Pluripotent stem cells are stem cells that has the potential to differentiate into any of the three germ layers: endoderm, mesoderm, or ectoderm.

Induced pluripotent cells (iPSCs) are special types of pluripotent cells that are derived from adult somatic cells upon ectopic expression of a set of transcription factors.

Adherent cells are the cells that are anchorage dependent and have to be cultured on a suitable substrate that is specifically treated to promote cell adhesion and growth.

Embryoid bodies (EB) are the three-dimensional aggregates formed in suspension by pluripotent stem cells (PSC), including embryonic stem cells (ESC) and induced pluripotent stem cells (iPSC), that mimic the structure of the developing embryo and have the potential to develop into cells of all three germ layers-ectoderm, mesoderm, and endoderm. EB differentiation is a common platform to generate specific cell lineages from PSCs.

Rosettes is the developmental signature of neuroprogenitors in cultures of differentiating embryonic stem cells; rosettes are radial arrangements of columnar cells that expresses early neuroectodermal markers such as Pax6 and Sox1 and are capable of differentiating into various region-specific neuronal and glial cell types in response to appropriate developmental cues.

Neurospheres are a spherical mass of cells produced by the continuous division of neural stem cells upon isolation and when grown in a suspension culture in a dish. Neurospheres provide a method to investigate NPCs in vitro.

Neurodegenerative disorder includes a wide range of conditions that result from progressive damage to cells and nervous system connections that are essential for mobility, coordination, strength, sensation, and cognition. Parkinson's disease is one such neurodegenerative disorder.

Confluency is the percentage of the growth medium area (surface area of the culture dish) covered by adherent cells. For example, 60% confluency indicates that 60 out of 100 parts of the growth surface are occupied by cells. Confluency is used as an indicator of cell growth and expansion during cell culture experiments.

Intranasal delivery is a non-invasive method where the therapeutic medications are insufflated through the patient's nose. Intranasal administration can bypass blood-brain barrier and rapidly deliver drugs from the nasal mucosa to the brain in a noninvasive way for the treatment of central nervous system disorders.

Pharmaceutically acceptable excipients include but are not limited to diluents, carriers and additives which are used for preparation of formulations.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

### Abbreviations

EDTA: Ethylenediaminetetraacetic acid
DMEM/F12: Dulbecco's Modified Eagle's medium/F12
SMAD: Suppressor of Mothers against Decapentaplegic
WNT: wingless-type MMTV (wingless/integrated)
IGF: insulin like growth factor
VTN: Vitronectin
MEM/alpha: Modified Eagle's medium alpha
KOSR: Knockout serum replacement
NEAA: Non-essential amino acids
ELISA: Enzyme linked immunosorbent assay
DPBS: Dulbecco's phosphate-buffered saline
MPTP: 1-methyl-4-phenyl-1,2,3,6-tetrahydropyridine
TGF beta: Transforming Growth Factor-beta
FGF: Fibroblast Growth Factor
FBS: Fetal bovine serum
HEPES: (4-(2-hydroxyethyl)-1-piperazineethanesulfonic acid)

The present disclosure is not to be limited in scope by the specific embodiments described herein, which are intended for the purposes of exemplification only. Functionally equivalent products, compositions, and methods are clearly within the scope of the disclosure, as described herein.

As discussed in the background section, neurodegenerative disorders like Parkinson's disease (PD) are characterized by onset in late adulthood, progressive clinical course, and dopaminergic neuronal loss in the central nervous system. Unfortunately, effective therapies and permanent cure for these diseases are almost non-existent. Compensating the loss of dopaminergic neurons with stem cell replacement is currently the most trusted strategy to combat the progressive clinical course of PD, in particular methods involving neural precursor cells. While both embryonic stem cells (ESCs) and induced pluripotent stem cells (iPSCs) have been used to derive NPCs, iPSCs are now preferred due to the availability of non-integrating vectors for their easy generation. Various protocols have been explored to derive neural precursor cells from pluripotent stem cells but generating a population of NPCs that is efficient enough to be transplanted and prevent the progression of PD is still a challenge.

To address the aforementioned problems, the present disclosure discloses an optimized cell culture protocol to generate an efficient population of iPSCs derived NPCs that are capable of being delivered in the intranasal delivery route to a PD induced in vivo model.

In an embodiment of the present disclosure, there is provided a cell culture protocol for obtaining NPCs (neural progenitor cells) from iPSCs (induced pluripotent stem cells) comprising the steps of: (a) generating embryoid bodies from a confluent culture of iPSCs, wherein the embryoid bodies are in non-adherent suspension culture, (b) plating the embryoid bodies on a culture dish coated with a suitable extracellular matrix in neural induction media (NIM) to obtain neuroectoderm lineage, wherein the NIM comprises at least one WNT pathway inhibitor and at least two SMAD pathway inhibitors, (c) culturing the neuroectoderm lineage for 6-8 days in neural proliferation media (NPM) for primary rosette formation, wherein the NPM does not comprise any inhibitor, (d) selecting and plating the primary rosettes as suspension culture on ultra-low attachment culture dish, (e) culturing the primary rosettes of step (d) in suspension culture for 4-5 days to facilitate primary neurosphere formation, (f) plating the primary neurospheres of step (e) on a culture dish coated with suitable extracellular matrix to obtain adherent primary neurospheres, (g) culturing the adherent primary neurospheres in the NPM for 5-6 days for secondary rosette formation, (h) selecting and plating the secondary rosettes as suspension culture on ultra-low attachment dish, (i) culturing the secondary rosettes of step (h) in suspension culture for 4-5 days to facilitate secondary neurosphere formation, (j) plating the secondary neurospheres on a culture dish coated with suitable extracellular matrix to obtain adherent secondary neurospheres, (k) culturing the adherent secondary neurospheres in the NPM for 5-7 days for tertiary rosette formation, and (l) enzymatically dissociating and plating the tertiary rosette to obtain the NPCs, wherein the confluent culture of iPSCs have a confluency in the range of 80%-90%.

In an embodiment of the present disclosure, there is provided a cell culture protocol for obtaining NPCs (neural progenitor cells) from iPSCs (induced pluripotent stem cells) as described herein, wherein generating the embryoid bodies comprises: (a) culturing the confluent iPSCs for 2-4 days in a growth media to form embryoid bodies, wherein the growth media comprises growth factors, organic molecules and lipid concentrates, (b) gradually subjecting the developing embryoid bodies from the growth media to the NIM by (i) culturing the developing embryoid bodies in a media composition comprising mTESR and NIM in a ratio of 70:30 for 24 hours, (ii) culturing the developing embryoid bodies obtained in step (i) in a media composition comprising mTESR and NIM in a ratio of 50:50 for 24 hours, and (iii) culturing the embryoid bodies obtained in step (ii) in the NIM for 24 hours followed by plating the embryoid bodies on a tissue culture dish.

In an embodiment of the present disclosure, there is provided a cell culture protocol for obtaining NPCs (neural progenitor cells) from iPSCs (induced pluripotent stem cells) as described herein, wherein the primary rosette formation comprises: (i) maintaining the neuroectoderm lineage in the NIM for 2-4 days; (ii) maintaining the neuroectoderm lineage obtained in step (i) in a media composition comprising NIM and NPM in a ratio of 50:50 for 24-48 hours for primary rosette formation, and wherein selecting the primary rosettes and the secondary rosettes in is carried out using neural rosette selection medium.

In an embodiment of the present disclosure, there is provided a cell culture protocol for obtaining NPCs (neural progenitor cells) from iPSCs (induced pluripotent stem cells) as described herein, wherein the at least one suitable extracellular matrix is selected from the group consisting of matrigel, laminin, vitronectin, fibronectin, collagen, poly-L-lysine, poly-L-ornithine, and combinations thereof. In another embodiment of the present disclosure, the at least one suitable extracellular matrix is matrigel.

In an embodiment of the present disclosure, there is provided a cell culture protocol for obtaining NPCs (neural progenitor cells) from iPSCs (induced pluripotent stem cells) as described herein, wherein the at least one WNT pathway inhibitor is selected from a group consisting of 4-(1,3,3a,4,7,7a-Hexahydro-1,3-dioxo-4,7-methano-2H-isoindol-2-yl)-N-8-quinolinyl-Benzamide, 5-(Phenylsulfonyl)-N-piperidin-4-yl-2-(trifluoromethyl)benzene sulfonamide, 2-(2′,3-Dimethyl-[2,4′-bipyridin]-5-yl)-N-(5-(pyrazin-2-yl)pyridin-2-yl) acetamide, 2-(4-(2-methylpyridin-4-yl)phenyl)-N-(4-(pyridin-3-yl)phenyl) acetamide, 8-Tetrahydro-2-[4-(trifluoromethyl)phenyl]-4H-thiopyrano[4,3-d]pyrimidin-4-one, and combinations thereof. In another embodiment of the present disclosure, the at least one WNT pathway inhibitors is 4-(1,3,3a,4,7,7a-Hexahydro-1,3-dioxo-4,7-methano-2H-isoindol-2-yl)-N-8-quinolinyl-Benzamide.

In an embodiment of the present disclosure, there is provided a cell culture protocol for obtaining NPCs (neural progenitor cells) from iPSCs (induced pluripotent stem cells) as described herein, wherein the at least two SMAD pathway inhibitors are selected from a group consisting of 4-[4-(1,3-benzodioxol-5-yl)-5-pyridin-2-yl-1H-imidazol-2-yl]benzamide, 4-(6-(4-(piperazin-1-yl)phenyl) pyrazolo[1,5-a]pyrimidin-3-yl) quinoline, 3-[(1R)-1-(2,6-dichloro-3-fluorophenyl) ethoxy]-5-(1-piperidin-4-ylpyrazol-4-yl)pyridin-2-amine, 5-chloro-2-N-[2-methoxy-4-[4-(4-methylpiperazin-1-yl) piperidin-1-yl]phenyl]-4-N-(2-propan-2-ylsulfonylphenyl)pyrimidine-2,4-diamine, and 9-ethyl-6,6-dimethyl-8-(4-morpholin-4-ylpiperidin-1-yl)-11-oxo-5H-benzo[b] carbazole-3-carbonitrile, 5-chloro-2-N-(5-methyl-4-piperidin-4-yl-2-propan-2-yloxyphenyl)-4-N-(2-propan-2-ylsulfonylphenyl)pyrimidine-2,4-diamine, and combinations thereof.

In an embodiment of the present disclosure, there is provided a cell culture protocol for obtaining NPCs (neural progenitor cells) from iPSCs (induced pluripotent stem cells) as described herein, wherein the at least one SMAD pathway inhibitor is either 4-[4-(1,3-benzodioxol-5-yl)-5-pyridin-2-yl-1H-imidazol-2-yl]benzamide or 4-(6-(4-(piperazin-1-yl)phenyl) pyrazolo[1,5-a]pyrimidin-3 -yl) quinoline. In another embodiment, wherein the at least two SMAD pathway inhibitor is a combination of 4-[4-(1,3-benzodioxol-5-yl)-5-pyridin-2-yl-1H-imidazol-2-yl]benzamide and 4-(6-(4-(piperazin-1-yl)phenyl) pyrazolo[1,5-a]pyrimidin-3-yl) quinoline.

In an embodiment of the present disclosure, there is provided a cell culture protocol for obtaining NPCs (neural progenitor cells) from iPSCs (induced pluripotent stem cells) as described herein, wherein the enzymatic dissociation of the tertiary rosette is carried out using an enzyme selected from the group consisting of Tryple select, Accutase, TrypLE, Gentle Cell Dissociation Reagent (GCDR), Dispase. In another embodiment, the enzyme is Accutase.

In an embodiment of the present disclosure, there is provided neural progenitor cells produced from the method as described herein.

In an embodiment of the present disclosure, there is provided a pharmaceutical composition comprising the neural progenitor cells produced from the method as described herein, and pharmaceutically acceptable excipients. In another embodiment, the pharmaceutically acceptable excipient is PBS (Phosphate buffer saline).

In an embodiment of the present disclosure, there is provided a pharmaceutical composition comprising the neural progenitor cells produced from the method as described herein, and wherein the composition is used in the manufacture of a nasal formulation for use in treatment of neurodegenerative disorders, and wherein the neurodegenerative disorder is selected from the group consisting of Parkinson's disease, Alzheimer's disease, and prion disease.

In an embodiment of the present disclosure, there is provided a method of treating a neurodegenerative disorder in a subject comprising administering the nasal formulation as described herein to the subject.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

The examples below describe the protocol disclosed in the present invention in detail. The protocol focuses on the differentiation of induced pluripotent stem cell, to form neural progenitor stem cells (NPCs). The cells obtained from the protocol were further verified by using immunofluorescence, flowcytometry and RT-PCR techniques. The cells display expression of important and relevant markers with respect to the days of differentiation. The features that differentiate the current protocols from other similar cell culture protocols include the induction factors to promote differentiation, the method of rosette selection, culturing tertiary neurospheres to ensure a pure population of NPCs and an efficient protocol for generating large number of NPCs.

The NPCs were further transplanted through the intra nasal route into PD induced in vivo models to assess its potential in disease recovery of the in vivo models.

### Example 1

### Materials and Methods

Composition of the different media as used in the protocol is described below:

mTESR medium was procured from Stem Cell Technologies. The media contains DMEM/F12 as the basal medium supplemented with FGF, TGF-beta, pipecolic acid, GABA, lipid concentrate, L-glutamine, NEAA, as active ingredients.

Composition of complete NIM medium—NIM here refers to Neural Induction Medium (NIM) having composition as below in Table 1.

TABLE 1

| Components | For 50 ml | |
|---|---|---|
| DMEM/F12 | 46.5 | ML |
| FBS (2%) | 1 | ml |
| Sodium Pyruvate | 500 | ul |
| Sodium Bicarbonate | 500 | ul |
| HEPES | 500 | ul |
| NEAA | 500 | ul |
| IWRI (wnt pathway | 50 | ul |
| inhibitor) | | |
| SB (ALK receptor | 50 | ul |
| inhibitor) | | |
| LDN (ALK receptor | 5 | ul |
| inhibitor) | | |
| IGF-1 | 5 | ul |
| N2 (media supplement) | 500 | ul |

Composition of complete NPM medium—NPM here refers to Neural Proliferation Medium (NPM) having composition as below in Table 2

TABLE 2

| Components | For 50 ml | |
|---|---|---|
| DMEM/F12 | 45.5 | ML |
| FBS (2%) | 1 | ml |
| Sodium Pyruvate | 500 | ul |
| Sodium Bicarbonate | 500 | ul |
| HEPES | 500 | ul |
| NEAA | 500 | ul |
| N2(media supplement) | 500 | ul |
| B27 | 1 | ml |

Neural Rosette Selection Medium was procured from Stem Cell Technologies.

Table 3 below provides different reagents and the vendors they were procured from:

TABLE 3

| Chemicals | Company | Catalog No |
|---|---|---|
| Matrigel (ES grade) | Corning | 354277 |
| DMEM-F12 | GIBCO | 10565-018 |
| DPBS | GIBCO | 14190-136 |
| Fetal Bovine Serum | Hyclone | SH3007103 |
| HEPES buffer | GIBCO | 15630-080 |
| Non-essential Amino | GIBCO | 11140-050 |
| Acids | | |
| Accutase | GIBCO | A11105-01 |
| Penicillin Streptomycin | GIBCO | 15140-122 |
| Trypsin EDTA | GIBCO | 25200-072 |
| PSC cryomedium | Thermo Fisher | A2644401 |
| IWR1 (WNT pathway | Sigma | I0161-25MG |
| inhibitor) | | |
| N2 media supplement | Thermo Fisher | 17502048 |
| IGF-1 | Stem cell technologies | 78022 |
| mTeSR | Stem cell technologies | 100-0274 |
| LDN193189 (SMAD | Stem cell technologies | 72142 |
| inhibitor) | | |
| SB431542 (SMAD | Stem cell technologies | 72234 |
| inhibitor) | | |
| Sodium bicarbonate | Thermo Fisher | 25080-094 |
| Sodium pyruvate | Thermo Fisher | 11360-070 |
| B27 supplement | Thermo Fisher | 17504044 |

### Example 2

Cell Culture Protocol for Generating NPCs from iPSCs in Detail

The cell culture protocol for generating NPCs from iPSCs (Working Cell bank of the iPSCs procured from RUCDR, NIH, Bethesda, USA) involves the following steps:
(a) Embryoid bodies were generated in the form of a non-adherent suspension culture from a confluent culture of iPSCs (about 80-90%). Embryoid bodies were maintained in mTESR medium for initial 2 days. On the third day, the embryoid bodies were maintained in 70:30 mTESR: NIM. The following day, the embryoid bodies were maintained in 50:50 mTESR: NIM. On the fifth day the embryoid bodies were maintained in complete NIM.
(b) On the sixth day, the embryoid bodies were then plated with 200-400 μl of media to ensure its uniform distribution and adherence on a culture dish coated with a suitable extracellular matrix in neural induction media (NIM) to obtain neuroectoderm lineage. The NIM comprises at least one WNT pathway inhibitor and at least two SMAD pathway inhibitors.
(c) The neuroectoderm lineage was cultured for 6-8 days in neural proliferation media (NPM) without any inhibitor for primary rosette formation.
(d) The primary rosette was selected using neuronal rosette selection medium. Before rosette selection, the neuroectoderm lineage was washed in plain DMEM and then incubated in the selection medium for 20 minutes. The incubation time might vary depending on the detachment status of the rosettes. The primary rosettes were plated as suspension culture on ultra-low attachment culture dish for 4-5 days to facilitate primary neurosphere formation.
(e) The primary neurospheres were then plated on a culture dish coated with suitable extracellular matrix to obtain adherent primary neurospheres.
(f) The adherent primary neurospheres were cultured in the NPM for 5-6 days for secondary rosette formation.
(g) The secondary rosette was selected and plated (as described earlier in step d) as suspension culture on ultra-low attachment dish, for 4-5 days to facilitate secondary neurosphere formation.
(h) The secondary neurospheres were plated on a culture dish coated with suitable extracellular matrix to obtain adherent secondary neurospheres, which were later cultured in the NPM for 5-7 days for tertiary rosette formation.
(i) The tertiary rosettes were enzymatically dissociated and plated to obtain the NPCs.

### Example 3

Characterization of iPSC Derived NPCs

Once the NPCs were produced using the above protocol, they were examined for appropriate morphology and expression of relevant markers.

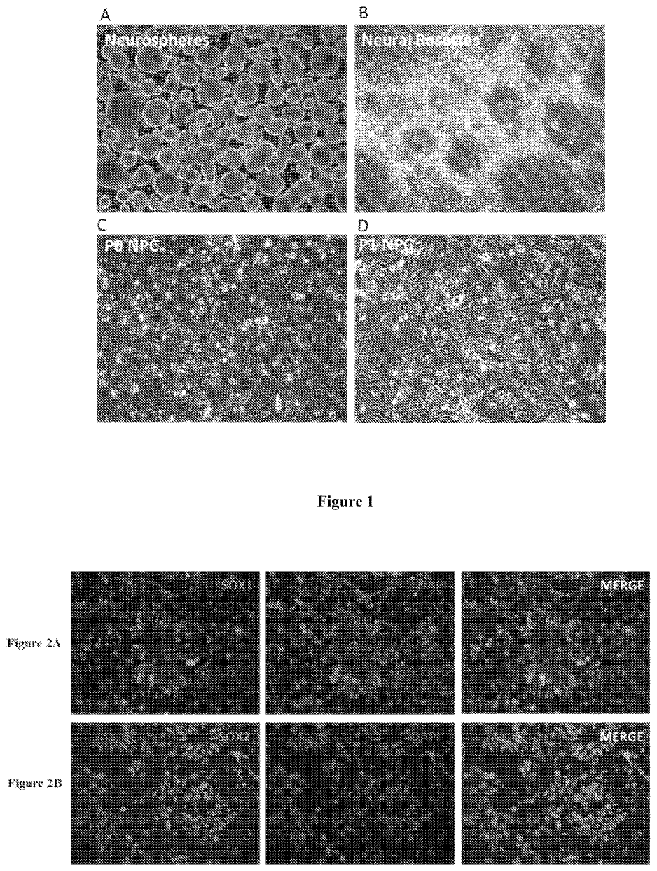

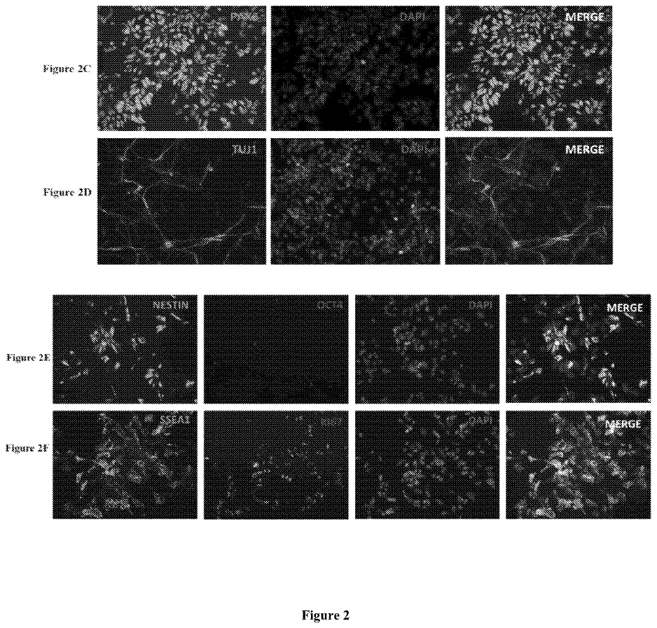

Phase Contrast Microscopy: The protocol used for the phase contrast microscopy is well known in the art. FIG. 1A-1D illustrates the phase contrast images displaying temporal changes in cellular morphology (1A—rosette formation and 1B—neurosphere formation) from undifferentiated iPSCs to neural progenitor cells (1C—Passage 0 (P0) NPCs; 1D—P1 NPCs).

Immunocytochemistry or Immunofluorescence study: For indirect immunofluorescence, cells were harvested at appropriate time points and fixed in 4% paraformaldehyde for 10 minutes at room temperature (RT) followed by washing three times in 1×PBS. Cells were then permeabilized for 5 minutes with 0.1% Triton X-100 (Sigma), then blocked for 1 hour at RT with 4% fetal bovine serum (FBS) and incubated with primary antibodies (list of antibodies as mentioned below in Table 4).

Followed by over-night primary antibody incubation at 12-18 hours, cells were washed with 1×PBS twice and probed with secondary antibodies for 1 hour at room temperature After secondary antibody treatment cells were washed thoroughly with 1×PBS. For antibodies specific against membrane epitope cells were not permeabilized. Cell nuclei were counterstained with DAPI (4′, 6-diamidino-2-phenylindole; 100 ng/ml) for 10 mins at RT and observed under fluorescent microscope (Olympus IX73).

Table 4 below depicts the list of antibodies used for the present study.

TABLE 4

| Vendor | CATALOG NO. | ANTIBODY NAME |
|---|---|---|
| Abcam | ab18102 | Mouse monoclonal Nestin |
| Abcam | ab195045 | Rabbit monoclonal Pax 6 |
| Abcam | ab18976 | Rabbit polyclonal Oct 4 |
| R&D systems | MAB2155 | Mouse monoclonal SSEA-1 |
| Abcam | ab171380 | Mouse monoclonal Sox 2 |
| Abcam | ab15580 | Rabbit polyclonal Ki67 |
| Abcam | ab8191 | Mouse monoclonal Ki67 |

FIG. 2A-2F depicts immunostaining of neural progenitor cells with different cell markers, i.e., A) SOX1, B) SOX2, C) PAX6, D) TUJ1, E) NESTIN and OCT4, F) SSEA1 and Ki67.

In vitro derived NPCs express key neural stem cells markers such as Pax6, Sox2, Nestin, Sox1, Oct4, Tuj1, Ki67 and SSEA1.

Real Time PCR quantification: Gene expression profile of markers relevant to NPCs was performed. For quantitative polymerase chain reaction (Q-PCR) analysis, RNA was extracted by RNeasy kit (Qiagen, Hilden, Germany, http://www.qiagen.com), and cDNA conversion was done using Revert Aid kit (Thermo Scientific) as per the manufacturer's instructions. 1 μg of RNA was used for cDNA conversion. Q-PCR was done with SYBER Green master mix (Life Technologies) using mRNA specific primers (sequences of primers as listed below as Table 5) on ABI 7900HT (Life Technologies) and was analysed by SDS 2.4 software.

Table 5 below lists out the sequences of the primers used in the present disclosure.

TABLE 5

| | |
|---|---|
| hPAX6 F | AGTTCTTCGCAACCTGGCTA |
| hPAX6 R | TGGTATTCTCTCCCCCTCCT |
| hFoxa2F | GAGCCCCATGTACGGGCAG |
| hFoxa2R | GGGGAAGAGGTCCATGATCCA |
| hOTX2 F | TGCAGGGGTTCTTCTGTGAT |
| hOTX2R | AGGGTCAGAGCAATTGACCA |
| hMAP2 F | CCCAGAAAAGCGCTCTTCTC |
| hMAP2 R | CTCTGCGAATTGGCTCTGAC |
| hNestin F | CAGCAACTGGCACACCTCAAGAT |
| hNestin R | AACCAAATGCAGCTTCAGCTTGG |
| hOct4F | CGAAAGAGAAAGCGAACCAG |
| hOct4R | GCCGGTTACAGAACCACACT |
| hSOX-2 F | ACCAGCTCGCAGACCTACAT |
| hSOX-2 R | TGGAGTGGGAGGAAGAGGTA |
| hβ-actin F | GTGGATCAGCAAGCAGGAGT |
| hβ-actin R | CTCGGCCACATTGTGAACTT |

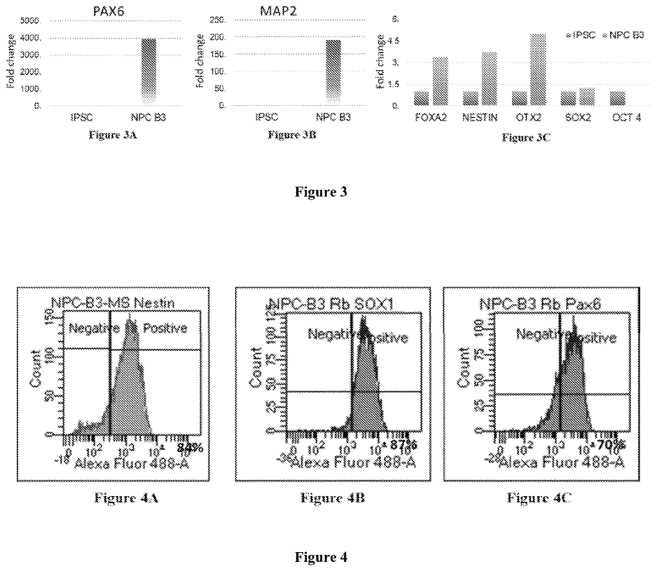

Normalization was performed based on the average of expression of constitutive gene β-actin. Study was performed on the final differentiated NPCs between day 25 to day 30. FIG. 3A-3C depicts RT-PCR results of gene expression profile of different cell markers such as Pax6 (FIG. 3A), MAP2 (FIG. 3B), Foxa2, Nestin, OTX2, Sox2 and Oct4 (FIG. 3C) of neural progenitor cells on differentiation.

Protein Expression profiling using Flowcytometry: Protein expression profile of markers relevant to NPCs was performed using flowcytometry. The protocol used for flow cytometry is well known in the art. FIG. 4A-4C depicts the protein expression profile of different cell markers such as, Nestin (FIG. 4A), Sox1 ((FIG. 4B) and Pax6 (FIG. 4C) of neural progenitor cells on differentiation. mRNA sequencing analysis

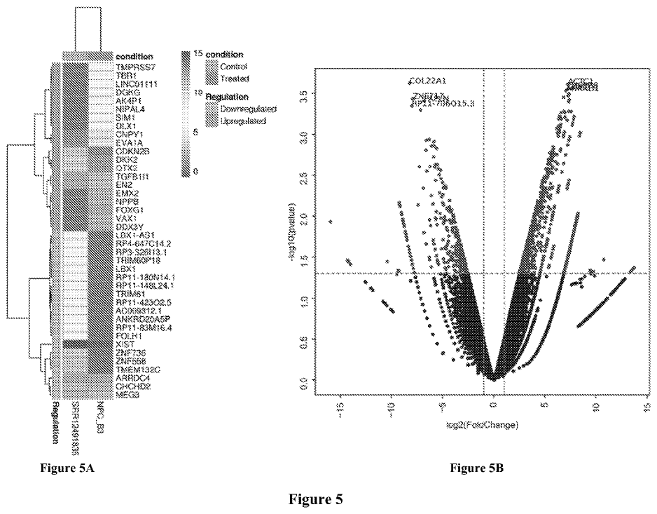

The differential gene expression signature of the differentiated NPCs obtained using the disclosed protocol when compared with the NPCs (i.e., the Control) obtained from cell culture protocols from prior art was determined using isolated mRNA. mRNA analysis as depicted in the interactive heat plot shows the top 20 genes that were upregulated and downregulated in Control Vs NPC-B3 cells (FIG. 5A). Further, from the volcano plot (FIG. 5B), which displays the log 2 differential gene expression analysis of NPC-B3 cells with respect to the Control, it can be inferred that genes such as ACTC1 were significantly upregulated (green points) and genes such as COL22A1 were significantly downregulated (red points).

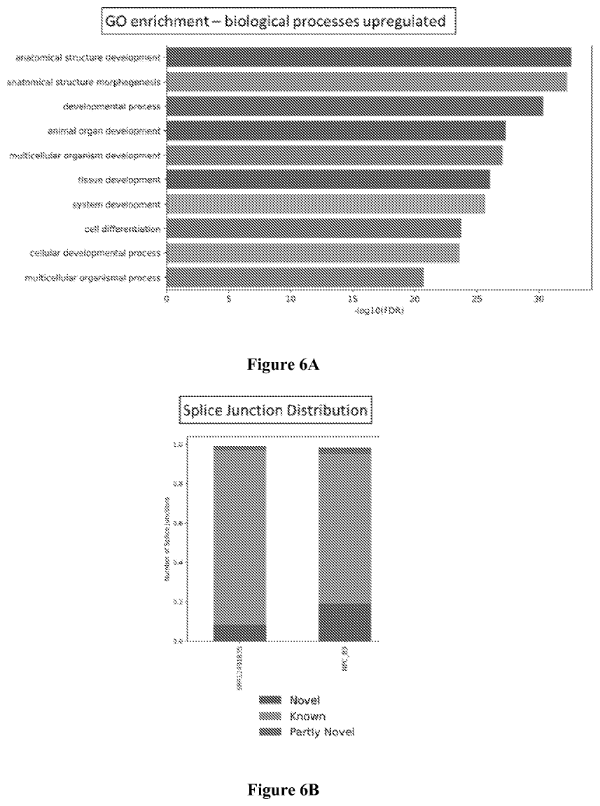

The gene ontology (GO) enrichment analysis was carried out for the upregulated gene signature of NPC-B3 cells with respect to the Control. As depicted in FIG. 6A, most of the upregulated genes of NPCs belonged to developmental biology processes such as anatomical structure development processes and anatomical structure morphogenesis processes (FIG. 6A). Further, higher number of novel as well as partly novel genes belonging to splice junction were found to be upregulated in NPCs when compared to the Control (FIG. 6B). Overall, the NPCs obtained using the disclosed protocol exhibited differential expression of genes involved in neurogenesis when compared to Control i.e. NPCs obtained from other protocols in prior art.

### Example 4

### Determination of the Biodistribution Potential of NSCs in Diseased Models Post Transplantation

The efficacy profile of the neural progenitor cells (obtained by the protocol of the present disclosure) was determined in MPTP rat models of PD. The model is most widely used in Parkinson's disease (PD) and employs the toxin, 1-methyl-4-phenyl-1,2,3,6-tetrahydropyridine (MPTP).

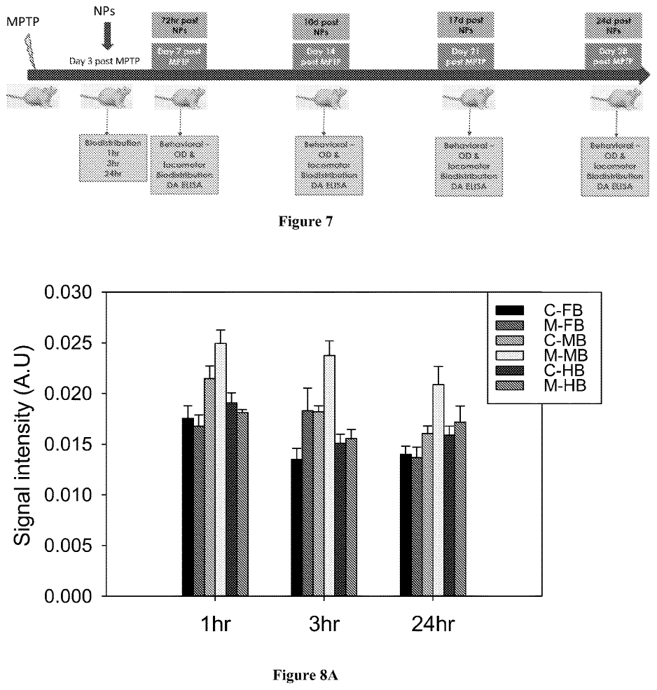

As shown in FIG. 7, the biodistribution potential of the transplanted NPCs was examined 1 hr, 3 hr, 24 hr, 72 hr, 10 day, 17 day and 24 day post NPCs transplantation in Control (non-MPTP treated rats) and MPTP treated rats. FIGS. 8 to 12, demonstrate the result of the aforesaid in-vivo experimentations.

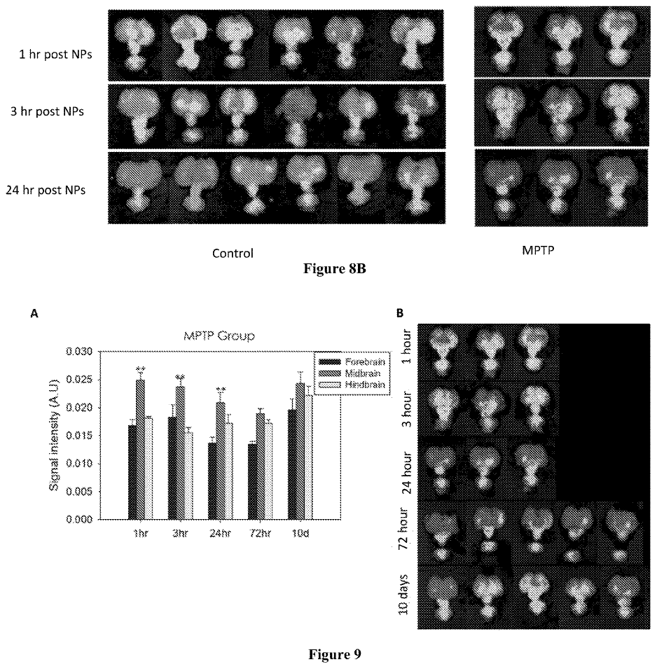

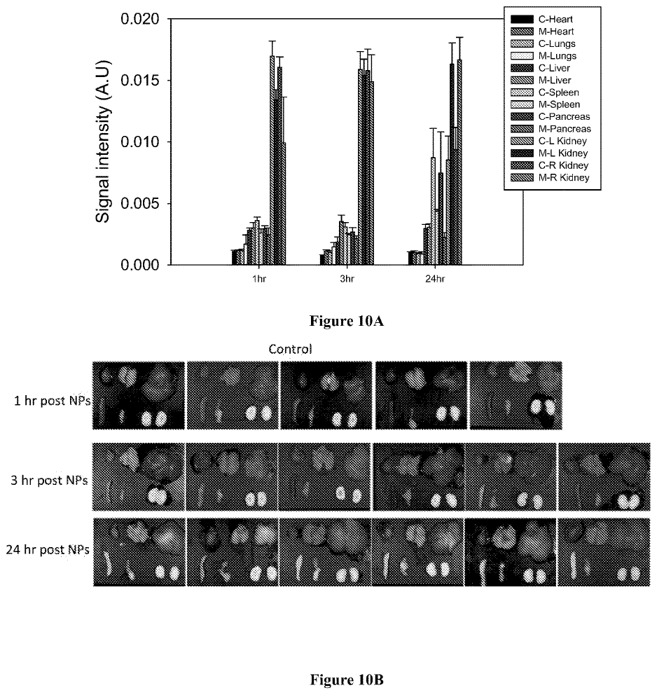

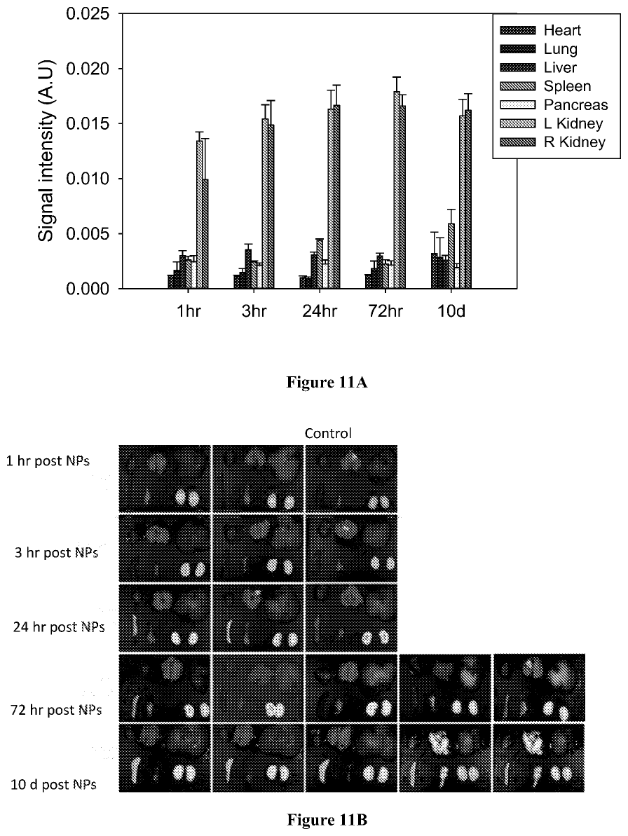

Increased signal intensity in different brain regions after intranasal transplantation with Near Infra-Red (NIR)-dye-treated NPCs is observed in MPTP-treated rats compared to control rats at each time point (FIG. 8B). Quantification of signal intensity in different brain regions shows a significant increase at both 1- and 3-hour(s) post transplantation of NIR-dye-treated NPCs across all brain regions. Till 24 hrs, the docking of cells was higher in the midbrain region (FIG. 8A). NPCs presence reduced at 72 hrs post transplantation however by day 10 a re-entry of the cells was observed in the MB probably due to neuroinflammatory signals of the midbrain (FIG. 9A-9B). Shown are representative images of n=5 experiments.

Transplanted NPCs entered the blood stream by 24 hr and the same was evidenced by their presence in other blood filtering organs (10A-10B). Flow of NPCs in the blood filtering organs, especially the kidneys, was observed from 1 hr itself. On day 10 post NPCs transplantation, an increase of cell docking was observed in spleen (FIG. 11A).

On 24 hr post NPCs transplantation, docking of NPCs in the kidneys was distinctly observed. At 10 days post NPCs transplantation, docking in spleen was significantly increased (FIG. 11A).

In control group NPCs transplantation showed docking in spleen and pancreas from 24 hr post transplantation suggesting some inflammatory trigger getting initiated by the NPCs (FIG. 11B). In MPTP group, the docking of NPCs was significantly lesser than control rats at 24 hr. However, at day 10 post NPCs transplantation an increase in docking was observed.

Till the day 10 of NPCs transplantation, no mortality of the rats in MPTP group was observed. Shown are representative images of n=5 . . . experiments.

### Example 5

### A Nasal Formulation of NPCs

The nasal formulation comprises of the iPSCs derived NPCs of the present disclosure resuspended in PBS (comprising nutrients such as glucose and supplements to promote cell viability) at the dosage of 1 million cells per injection per rat.

### Example 6

### Rescue Potential of the Transplanted NPCs

The rescue potential of the transplanted NPCs was determined by examining the behavioral changes of the rats such as of olfactory discrimination (premotor behaviour) and locomotor activity (motor behaviour).

### Olfactory Discrimination

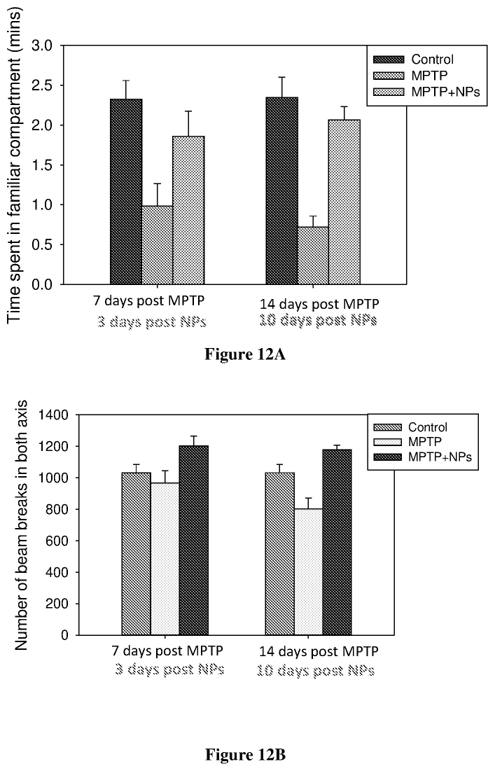

Distinct improvement in olfactory discrimination was observed from 72 hrs post NP administration. The improvement observed post 72 hrs of NPCs transplantation was maintained even on day 10 post NPCs transplantation (FIG. 12A).

### LocoMotor Behaviour

Locomotor impairment initiates from day 14 post MPTP, and a significant recovery in locomotor function was observed upon NPCs transplantation (FIG. 12B).

Collectively, NPCs have the potential to rescue a PD induced rat model in terms of premotor and motor parameters.

### Advantages of the Present Disclosure

The present disclosure discloses an optimized cell culture protocol for generating NPCs from iPSCs and further, discloses a method to rescue PD induced rat models using a nasal formulation comprising the NPCs. The present disclosure has the following advantages:
(a) These NPCs are produced by mimicking the in vivo milestones of neurogenesis during embryonic development. Hence the molecular characteristics of these de novo cells are similar to that of native or primary NPCs. Further, the protocol is short and does not require many recombinant growth factors (only small molecules) which makes it cost effective. Successful freeze-thaw of these cells make the process scalable and affordable.
(b) In comparison to fully mature post-mitotic specialized neuronal cell types, NPCs have demonstrated better survival and engraftment resulting in more efficient rescue of the disease symptoms in rats.
(c) MPTP infusion directly into the rat striatum and through intranasal route causes Parkinsonian symptoms with depletion of striatal dopamine and non-motor and motor impairments. This cannot be achieved systemically or intraperitoneally.

1. A cell culture protocol for obtaining NPCs (neural progenitor cells) from iPSCs (induced pluripotent stem cells) comprising the steps of:
(a) generating embryoid bodies from a confluent culture of iPSCs, wherein the embryoid bodies are in non-adherent suspension culture,
(b) plating the embryoid bodies on a culture dish coated with a suitable extracellular matrix in neural induction media (NIM) to obtain neuroectoderm lineage, wherein the NIM comprises at least one WNT pathway inhibitor and at least two SMAD pathway inhibitors,
(c) culturing the neuroectoderm lineage for 6-8 days in neural proliferation media (NPM) for primary rosette formation, wherein the NPM does not comprise any inhibitor,
(d) selecting and plating the primary rosettes as suspension culture on ultra-low attachment culture dish,
(e) culturing the primary rosettes of step (d) in suspension culture for 4-5 days to facilitate primary neurosphere formation,
(f) plating the primary neurospheres of step (e) on a culture dish coated with suitable extracellular matrix to obtain adherent primary neurospheres,
(g) culturing the adherent primary neurospheres in the NPM for 5-6 days for secondary rosette formation,
(h) selecting and plating the secondary rosettes as suspension culture on ultra-low attachment dish,
(i) culturing the secondary rosettes of step (h) in suspension culture for 4-5 days to facilitate secondary neurosphere formation,
(j) plating the secondary neurospheres on a culture dish coated with suitable extracellular matrix to obtain adherent secondary neurospheres,
(k) culturing the adherent secondary neurospheres in the NPM for 5-7 days for tertiary rosette formation, and
(l) enzymatically dissociating and plating the tertiary rosettes to obtain the NPCs.

2. The cell culture protocol as claimed in claim 1, wherein the confluent culture of iPSCs have a confluency in the range of 80%-90%.

3. The method as claimed in claim 1, wherein generating the embryoid bodies in step (a) comprises:
(a) culturing the confluent iPSCs for 2-4 days in a growth media to form embryoid bodies, wherein the growth media comprises growth factors, organic molecules, and lipid concentrates,
(b) gradually subjecting the developing embryoid bodies from the growth media to the NIM by (i) culturing the developing embryoid bodies in a media composition comprising mTESR and NIM in a ratio of 70:30 for 24 hours, (ii) culturing the developing embryoid bodies obtained in step (i) in a media composition comprising mTESR and NIM in a ratio of 50:50 for 24 hours, and (iii) culturing the embryoid bodies obtained in step (ii) in the NIM for 24 hours followed by plating the embryoid bodies on a tissue culture dish.

4. The method as claimed in claim 1, wherein the primary rosette formation in step (c) comprises:
(i) maintaining the neuroectoderm lineage in the NIM for 2-4 days;
(ii) maintaining the neuroectoderm lineage obtained in step (i) in a media composition comprising NIM and NPM in a ratio of 50:50 for 24-48 hours for primary rosette formation.

5. The method as claimed in claim 1, wherein selecting the primary rosettes in step (d) and the secondary rosettes in step (h) is carried out using neural rosette selection medium.

6. The cell culture protocol as claimed in claim 1, wherein the at least one suitable extracellular matrix is selected from the group consisting of matrigel, laminin, vitronectin, fibronectin, collagen, poly-L-lysine, poly-L-ornithine, and combinations thereof.

7. The cell culture protocol as claimed in claim 1, wherein the at least one WNT pathway inhibitor is selected from a group consisting of 4-(1,3,3a,4,7,7a-Hexahydro-1,3-dioxo-4,7-methano-2H-isoindol-2-yl)-N-8-quinolinyl-Benzamide, 5-(Phenylsulfonyl)-N-piperidin-4-yl-2-(trifluoromethyl)benzene sulfonamide, 2-(2′,3-Dimethyl-[2,4′-bipyridin]-5-yl)-N-(5-(pyrazin-2-yl) pyridin-2-yl) acetamide, 2-(4-(2-methylpyridin-4-yl)phenyl)-N-(4-(pyridin-3-yl)phenyl) acetamide, 8-Tetrahydro-2-[4-(trifluoromethyl)phenyl]-4H-thiopyrano[4,3-d]pyrimidin-4-one, and combinations thereof.

8. The cell culture protocol as claimed in claim 1, wherein the at least two SMAD pathway inhibitors are selected from a group consisting of 4-[4-(1,3-benzodioxol-5-yl)-5-pyridin-2-yl-1H-imidazol-2-yl]benzamide, 4-(6-(4-(piperazin-1-yl)phenyl) pyrazolo[1,5-a]pyrimidin-3-yl) quinoline, 3-[(1R)-1-(2,6-dichloro-3-fluorophenyl) ethoxy]-5-(1-piperidin-4-ylpyrazol-4-yl)pyridin-2-amine, 5-chloro-2-N-[2-methoxy-4-[4-(4-methylpiperazin-1-yl) piperidin-1-yl]phenyl]-4-N-(2-propan-2-ylsulfonylphenyl)pyrimidine-2,4-diamine, and 9-ethyl-6,6-dimethyl-8-(4-morpholin-4-ylpiperidin-1-yl)-11-oxo-5H-benzo[b]carbazole-3-carbonitrile, 5-chloro-2-N-(5-methyl-4-piperidin-4-yl-2-propan-2-yloxyphenyl)-4-N-(2-propan-2-ylsulfonylphenyl) pyrimidine-2,4-diamine, and combinations thereof.

9. The method as claimed in claim 1, wherein the enzymatic dissociation of tertiary rosette in step (1) is carried out using an enzyme selected from the group consisting of Accutase, Tryple select, TrypLE, Gentle Cell Dissociation Reagent (GCDR), Dispase.

10. The neural progenitor cells produced by the method as claimed in claim 1.

11. A pharmaceutical composition comprising the neural progenitor cells as claimed in claim 10 and pharmaceutically acceptable excipients.

12. A nasal formulation comprising the pharmaceutical composition as claimed in claim 11.

13. (canceled)

14. A method of treating a neurodegenerative disorder in a subject comprising administering the nasal formulation as claimed in claim 12 to the subject.

15. The method of claim 14, wherein the neurodegenerative disorder is selected from the group consisting of Parkinson's disease, Alzheimer's disease, and prion disease.
